(19) United States
(12) Patent Application Publication
(10) Pub. No.: US 2007/0105097 A1
(43) Pub. Date: May 10, 2007

(54) Method for comparing gene expression level

(75) Inventor: Guohua Zhou, Nanjing (CN)

Correspondence Address: SUITER WEST SWANTZ PC LLO, 14301 FNB PARKWAY, SUITE 220, OMAHA, NE, 68154, US

(73) Assignee: Huadong Rearch Institute for Medicine and Biotechn, Jiangsu (CN)

(21) Appl. No.: 10/523,953

(22) PCT Filed: Aug. 8, 2003
(86) PCT No.: PCT/CN03/00648

§ 371 Date: Sep. 29, 2005

(30) Foreign Application Priority Data
Aug. 9, 2002 (CN) 02138093.7

(51) Int. Cl. C12Q 1/68; C12P 19/34

(52) U.S. Cl. 435/6; 435/91.2

(57) ABSTRACT

The present invention relates to a method for comparing and determining the gene expression level, which is very important in disease-related gene screening, early clinical diagnosis and medicine development. The present invention utilizes quantitative characteristics of bioluminescence analysis to compare gene expression levels of different individuals or samples in order to search for disease-related genes. The specific steps include: (i) labeling the mRNA of a given gene from different sources through a suitable method, and mixing the labeled fragments to obtain PCR templates; (ii) performing a polymerase chain reaction using source-specific primers and a gene-specific primer; and (iii) detecting the sequence of the amplified DNA fragments by bioluminescence analysis, the base sequence in the sequencing profile representing the gene source, and the signal intensity of each base representing the gene expression level from the corresponding source.

### FIELD OF THE INVENTION

This invention relates to a method for quantitatively determining a DNA fragment in a DNA mixture, and specifically to a method for using bioluminescence assay to simultaneously determine gene expression level from different sources, which may be used for the comparative analysis of the expression level of a given gene in different sources.

### BACKGROUND OF THE INVENTION

With the progress of the molecular biology, the whole genomic DNA of several tens of biological species have been sequenced, and the human genome project will be finished soon (Venter J C, Adams M D, Myers E W, Li P W, Mural R J, Sutton G G, Smith H O, et al.: The sequence of the human genome. Science 2001; 291(5507):1304-51; McPherson J D, Marra M, Hillier L, Waterston R H, Chinwalla A, Wallis J, Sekhon M, Wylie K, et al.: A physical map of the human genome. Nature 2001; 409(6822):934-41). The first step of the human genome project is to analyze the structure of genome. The second step is to clarify gene functions coded in genomes, which includes understanding the distribution of mRNA, which is the transcription product of a gene, as well as the amount, the function and the distribution of proteins, which are the expressed products of mRNAs, in a cell or in the tissue of an organ. Comparative analysis of gene expression profiling can be used to find the functions of unknown genes and to clarify the interactions between genes or between proteins (Matsubara, K., and K. Okubo. 1993. cDNA analyses in the human genome project. Gene 135: 265-74). Therefore, gene expression profiling is becoming one of the main research areas in DNA analysis. In clinical medicine, disease-related genes can be found by quantitatively comparing the gene expression levels of given genes between different sources, such as different individuals (e.g. healthy persons and patients) or different organs (e.g. heart, lung or brain). These disease-related genes are very helpful for creating disease-specific drug targets. In the medicine of disease-prevention, it is very difficult to use regular methods to timely diagnose multi-gene related disease, such as cancer, diabetes and obesity. However, the expression profiling of disease-related genes in the related organs can be used to prevent a disease by predicting the risk of suffering from a disease. Furthermore, in the field of molecular biology research, gene expression profiling is helpful for finding new functional genes to upgrade a biological species. Up to now, there are various methods used for gene expression profiling, including Northern blotting (Kawasaki, E. S., S. S. Clark, M. Y. Coyne, S. D. Smith, R. Champlin, O. N. Witte, and F. P. McCormick. 1988. Diagnosis of chronic myeloid and acute lymphocytic leukemias by detection of leukemia-specific mRNA sequences amplified in vitro. Proceedings of the National Academy of Sciences of the United States of America 85: 5698-702), real-time PCR (RT-PCR) (Karet, F. E., D. S. Charnock-Jones, M. L. Harrison-Woolrych, G. O'Reilly, A. P. Davenport, and S. K. Smith. 1994. Quantification of mRNA in human tissue using fluorescent nested reverse-transcriptase polymerase chain reaction. Anal. Biochem. 220: 384-90), sequencing (Velculescu, V. E., L. Zhang, B. Vogelstein, and K. W. Kinzler. 1995. Serial analysis of gene expression. Science 270: 484-7; Powell, J. 2000. SAGE. The serial analysis of gene expression. Methods in Molecular Biology 99: 297-319), and microarray (Schena, M., D. Shalon, R. W. Davis, and P. O. Brown. 1995. Quantitative monitoring of gene expression patterns with a complementary DNA microarray. Science 270: 467-70; Hegde, P., R. Qi, K. Abernathy, C. Gay, S. Dharap, R. Gaspard, J. E. Hughes, E. Snesrud, N. Lee, and J. Quackenbush. 2000. A concise guide to cDNA microarray analysis. Biotechniques 29: 548-50, 552-4, 556 passim; Ferguson, J. A., T. C. Boles, C. P. Adams, and D. R. Walt. 1996. A fiber-optic DNA biosensor microarray for the analysis of gene expression. Nature Biotechnology 14: 1681-4).

Northern blotting is a classical method which is mainly used for analyzing the expression level of several or tens of genes. However, the detection procedure is very complicated and operators for the test have to be well-trained. In addition, it uses radioactive substance which is harmful to both operators and the environment. Furthermore its sensitivity is very low, and it is impossible to detect small amount of gene expression products.

In RT-PCR, mRNA is converted into DNA by reverse transcription, followed by PCR amplification using a gene-specific primer and a polyadenine nucleotide (polyA) primer, and the amplified DNA fragments are separated by electrophoresis. Gene expression information and the expressed level of each gene are obtained from the intensities of the electrophoretic bands of each sample. Although this method has a high sensitivity, the reproducibility is poor. In addition, the quantification is not so satisfactory even if the internal standard is used. This is because the linear relationship between the amount of PCR products and the amount of DNA templates is poor. The detection results cannot reflect the gene expression level faithfully.

Sequencing is a method based on the large scale of base sequence determination of cDNAs for calculating gene expression level, which mainly includes body mapping and serial analysis of gene expression (SAGE). Both methods are accurate, but body mapping needs DNA sequencer to determine the frequency of each gene-specific sequence in the sample by sequencing cDNAs, each representing a gene. The drawbacks include a large workload and a need for expensive instruments. SAGE is a modified sequencing method. At first, the beaded cDNAs are digested into fragments with sticky ends by a restriction endonuclease, followed by dividing the digested beaded-cDNA into two equal parts. Two specific DNA linkers, liker-A and liker-B with sticky overhands, are added for the ligation reaction. These ligated products are then cut into small fragments with 9-12 bp tag bases by IIs-type restriction enzyme that has an ability to cut the DNA fragments into a fragment with a given length from the recognition cutting site. These short tag fragments are used to identify the gene type. Two aliquots are mixed and these tags are ligated tail to tail by ligase. PCR amplification is performed by adding primers with the sequences identical to the parts of the sequence in liker-A and -B. Each PCR fragment contains a ditag which represents two genes. These PCR products are cut into the fragments containing a four-base sticky cutting site by the foregoing restriction enzyme, followed by cloning. Each clone contains 10 to 50 gene-tags that are divided by a four-base interval (the specific recognition sequence of the restriction endonuclease). Finally, the clone is sequenced. The expression amount of each gene is calculated by the abundance of a tag sequence in the whole sequence of the cloned products. Although it is not necessary to determine the sequence of each cDNA, it is labor-intensive and the operation procedure is very complicated. Also an expensive sequencer is required. It is difficult for an average laboratory to perform a SAGE analysis. Thus, SAGE is not commonly used.

DNA microarray is a method using cDNAs or oligonucleotide fragments (20-30 bp) attached on solid matrices to hybridize the labeled cDNAs transcribed from mRNA in biomaterials. A single chip can hybridize samples from two different sources, and different samples are labeled with different fluorescent groups. The relative amounts of the expressed genes from two different sources are obtained by comparing the signal intensities from two different dyes in each spot on the microarray. The drawbacks include low sensitivity, poor quantification and the need of special software for processing data. Moreover, the detection instrument is very expensive.

Pyrosequencing is a method for DNA sequencing based on bioluminometric assay (Ronaghi, M., M. Uhlen, and P. Nyren. 1998. A sequencing method based on real-time pyrophosphate. Science 281: 363, 365). As only 10-30 bases are sequenced at a time, it is mainly applied for SNP detection (Ahmadian, A., B. Gharizadeh, A. C. Gustafsson, F. Sterky, P. Nyren, M. Uhlen, and J. Lundeberg. 2000. Single-nucleotide polymorphism analysis by pyrosequencing. Anal Biochem 280: 103-10). This method has many advantages, including a good quantitative capability, high sensitivity and simple operation. In addition, it does not require electrophoresis, labeling reactions, use of a laser, or use of special or expensive reagents. Only simple instrumentation is needed for the detection. Pyrosequencing is based on quantitative PPi detection by luminometric assay for sequencing, whose measurement principle is as follows.

PPi in a quantity equimolar to the amount of incorporated nucleotide is released from the polymerase-catalyzed extension reaction of the single-stranded DNA annealed with a sequencing primer if a complementary dNTP is added. PPi is converted into ATP by the catalysis of ATP sulfurylase. Light is emitted by the reaction of the ATP with luciferin by catalysis of luciferase. The visible light is detected by a charge-coupled device (CCD) camera or photomultiplier tube (PMT). As the signal intensities are proportional to the amount of PPi, the sequence of a target DNA is determined by the species of the dispensed dNTP and the relative peak intensities.

However the technique cannot be used for gene expression profiling directly.

### SUMMARY OF THE INVENTION

To overcome the drawbacks in the methods described above for the gene expression profiling, this invention proposes a sensitive, quantitative, inexpensive and feasible method for gene expression analysis.

The method of the present invention is as follows.

A method for comparing gene expression level, characterized in that the method includes:

(a) labeling mRNA from different sources with a suitable method, and mixing the labeled mRNA fragments equally to obtain a template for polymerase chain reaction (PCR);

(b) performing a polymerase chain reaction using source-specific primers and a gene-specific primer; and

(c) detecting a sequence of amplified DNA fragments with bioluminescence analysis, a base type and a signal intensity in a sequencing profile representing a gene source and a relative expression level, respectively.

The foregoing described terminology of “mRNA from different sources” represents the expressed mRNA of a given gene from different individuals of a species, or is the expressed mRNA of a given gene from different organs of an individual, or is the expressed mRNA of a given gene of the same species at different states of chemical stimulation or physical stimulation.

The foregoing described terminology of “source-specific primers” represents primers including identical base species and base number but different base order, and each primer represents a gene source.

The foregoing described terminology of “a suitable method” represents methods to distinguish the gene source by a DNA fragment with a suitable length. The first method is to distinguish the gene sources by performing a reverse transcription-polymerase chain reaction (RT-PCR) to obtain complementary DNA (cDNA) fragments of a given gene in each of the sources, followed by digesting cDNA into fragments with a suitable length using a restriction enzyme, and then ligating each of the digested cDNA fragments with a selective adapter, where different adapter corresponds to mRNA from different sources. The second method is to distinguish the gene sources by synthesizing the first strand of the complementary DNA (cDNA) fragments of mRNA samples from each of the sources using polythymine primers fixed on microsphere's surface, and then synthesizing the complementary second strand cDNA using anchored primers containing the sequences corresponding to gene sources in the 5′-terminal region, where 5′-end is used for identifying different sources of a given gene. The third method is to distinguish the gene sources by preparing the first strand of the complementary DNA (cDNA) fragments of mRNA samples from each of the sources by directly hybridizing anchored primers containing the sequences corresponding to gene sources in the 5′-terminal region with mRNA, where the construction of the 5′-terminal region of the anchored primers is the same as that in the second method.

Various drawbacks and problems in the existing methods used for comparing gene expression levels between different individuals are solved by this invention, which is based on the quantitative detection and comparison of the expression level of mRNA from different sources with bioluminometric assay based on the principle of pyrosequencing. It can be used to find disease-related genes for clinical diagnosis. This invention has the advantages of a high sensitivity, accurate quantification, a low running cost and a simple procedure for manipulation.

### BRIEF DESCRIPTION OF THE DRAWINGS

FIG. 1 illustrates the detection principle of the present invention;

FIG. 2 is a schematic diagram illustrating the procedure for detecting gene expression levels in two different sources;

FIG. 3 depicts the structure of DNA adapters;

FIG. 4 is a schematic diagram illustrating a reaction module;

FIG. 5 is a spectrum for comparing the gene expression levels from two sources using bioluminometric assay;

FIG. 6 a schematic diagram illustrating the structure of a device using a 96-well plate for simultaneously detecting expression levels of multiple genes;

FIG. 7 shows a procedure for determining an average gene expression level in two pooled samples; and

FIG. 8 shows the sequencing results of the sample in Embodiment 1 of the invention by using the pyrophosphate (PPi) detection solution without apyrase.

### DETAILED DESCRIPTION OF THE INVENTION

The present invention is further explained in combination with the attached drawings.

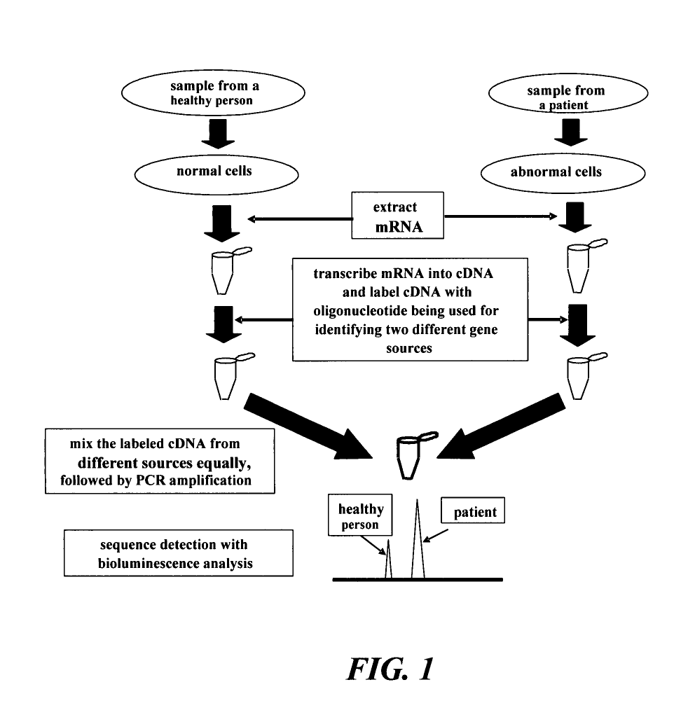

The present invention includes three steps: (1) transcription and labeling of cDNA from different sources; (2) preparation of PCR templates by equally mixing the labeled cDNA from different sources; (3) sequencing with a method based on bioluminometric assay. The detection principle is described in FIG. 1.

The first key point of this invention is how to use a suitable method to label cDNA from different sources for keeping a proportional amplification by PCR before PCR amplification. There are several strategies for implementing this: (1) Double-stranded cDNA transcribed from mRNA is digested into several fragments by a restriction endonuclease, followed by ligating the digested dsDNA fragments with source-specific adapters, each being composed of identical base species and base number but different base order. After the ligated cDNA samples are equally mixed together, PCR amplification is carried out; (2) The first strand cDNA is synthesized after the hybridization of mRNA with polythymine primers fixed on microsphere's surface. The complementary strand of the first strand cDNA is synthesized by a gene-specific primer with an anchored sequence for identifying the source of each cDNA in the 5′-terminal region. The template of PCR amplification is prepared by separating DNA strands with microspheres. Finally, PCR amplification is performed by using a gene-specific primer and primers having the same sequence as the part of the anchored sequence for identifying gene source; and (3) The first strand cDNA is synthesized after the hybridization of mRNA with a gene-specific primer with an anchored sequence for identifying the source of each cDNA in the 5′-terminal region. After the cDNA samples from various sources are equally mixed together, PCR amplification is carried out. A part of anchored sequence for identifying the source of each cDNA is used as a primer of PCR.

The second key point is how to extract the gene source information from the base sequence by pyrosequencing and how to extract the gene expression amounts from signal intensities in a pyrosequencing profile. In this invention, it is realized by introducing several bases with different sequences into cDNAs (templates of PCR amplification) before PCR amplification. The introduced sequence comprises identical base species and base number but different base order. Therefore, PCR amplification with the equal proportionality is realized by using the mixture of cDNAs labeled by the method described above as templates. Finally, sequencing reaction is carried out by adding the mixture of primers corresponding to each of cDNA sources. In the sequencing result, base species in the sequence represents the source of cDNA, and its intensity represents the expression level of the gene from the corresponding source. This is further explained by the following examples.

### EMBODIMENT 1

### Comparison of Gene Expression Levels from Two Individuals

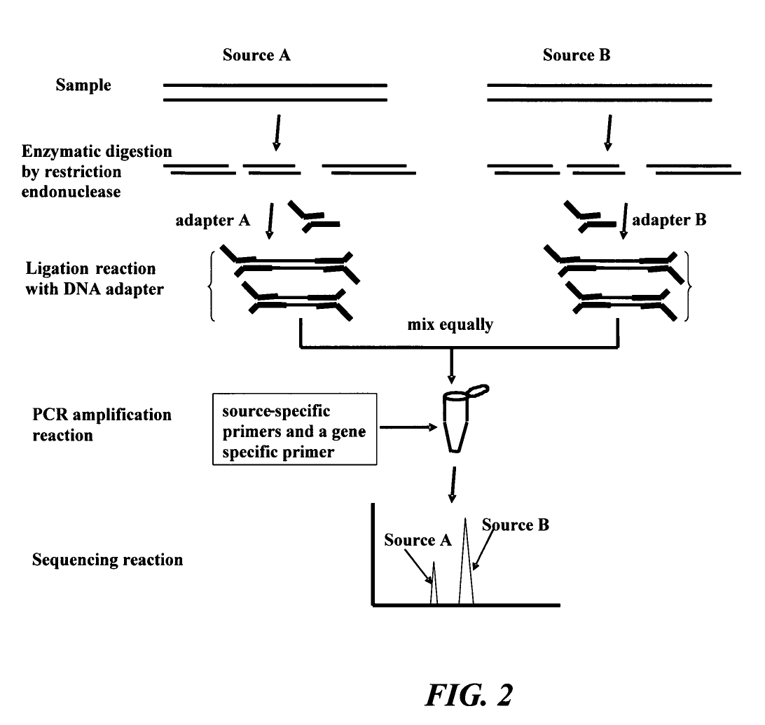

This example describes a method of PCR using the templates produced by equally mixing adapter-ligated cDNAs from source-A and source-B together. Before adapter ligation, each cDNA is digested into fragments with a restriction endonuclease. FIG. 2 is a schematic diagram illustrating the procedure for detection. Here human P53 gene is used as an example for the illustration. The extraction of mRNA is carried out by the standard method using Gibico TRI20LLS-Reagent™ kit.

1. Preparation of cDNA Samples (cDNA Sample from Each of Sources is Prepared Respectively)

The first strand of cDNA is synthesized using the kit of Gibico Super Script™ Preamplification System for First Strand cDNA Synthesis. 0.5 μg Oligo (dT)12-18 and 5.5 μl H₂O is added into 1 μg mRNA from source-A or source-B, and the solution is mixed homogeneously. After 10-min incubation at 0° C., a prepared mixture containing 4 μl of 5× buffer (I), 2 μl of 0.1 M dithiothreitol, 1 μl of 10 mM dNTPs, 2 μl of H₂O and 200 U/μl of reverse transcriptase is added to the above template solution and the resulting mixture is incubated at 42° C. for 50 min and 70° C. for 15 min. After the reaction is finished, the solution is kept at 10° C. before use.

Ten μl of 10× ligation buffer, 70 μl of H₂O, 1 μl of 1 mM dNTPs mixture, 50 U polymerase 1 and 10 U DNA ligase are added into the foregoing-described reaction mixture. After the mixture is homogenized, 2U RNase H is added and vibrated. The double-stranded cDNA is produced by incubating the mixture at 10° C. for 2 hours and 70° C. for 15 min. The solution is kept at −20° C. for future use.

Enzymatic digestion is performed by adding 20 μl of 10× digestion buffer (200 mM Tris-HCl (pH8.5), 100 mM MgCl₂, 10 mM dithiothreitol (DTT) and 1 M KCl), 30 μl of H₂O, and 60 U of restriction endonuclease Mbo I into 150 μl of the foregoing-described prepared cDNA solutions, and the mixture is incubated at 37° C. over night. Deactivation of restriction endonuclease Mbo I is performed by a 15-min incubation at 70° C.

2. Preparation of DNA Adapter

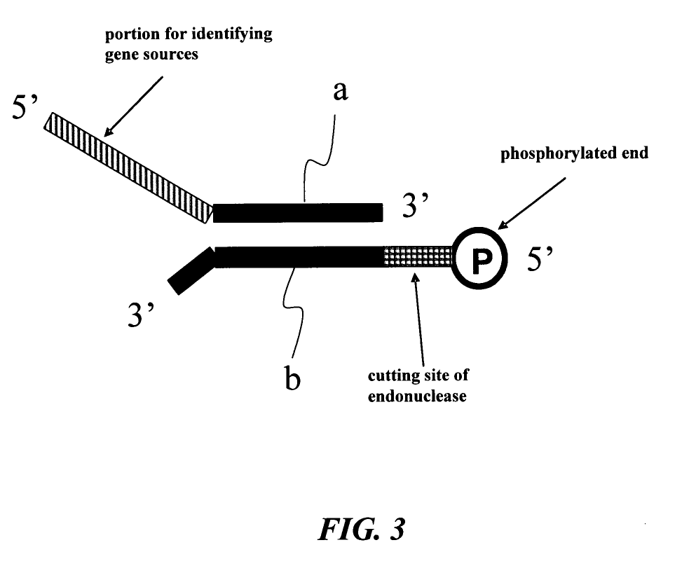

In this EMBODIMENT 1, DNA adapters are used to identify the source of the expressed gene. Firstly, the double-stranded cDNAs are cut into a cuneal structure with a four-base overhang of “ctag” in the 5′-end by the restriction endonuclease, Mbo I, followed by the ligation with DNA adapters. FIG. 3 depicts the structure of the DNA adapter that is composed of two partly complementary strands. The arm in 5′-terminus of strand “a” is used to identify the source of a gene. The nucleotide species and the number of each nucleotide in the 5′ terminus of each adapter are identical, but the order of each nucleotide is different. To block the extension reaction from strand “b” during PCR amplification, the 3′-end of the strand “b” is designed to be non-complementary to the strand “a”. There is a four-base overhand with an endonuclease-recognition sequence of “gatc” in the 5′-terminus of “b”, and the 3′-terminus of “a” is phosphorylated.

DNA adapter-A and adapter-B are used for identifying the source-A and source-B of the given gene, respectively. All of the sequences of adapter-A and -B are identical except the sequence of 5′-terminal region of strand “a”.

P-1 is assigned as the strand “a” of adapter-A, and its sequence is:

(SEQ ID NO: 1)

| | |
|---|---|
| P-1: | 5′-CCCCACTTCTTGTTCTCTCATCAGGCGCATCACTCG-3′ |

P-2 is assigned as the strand “a” of adapter-B, and its sequence is:

(SEQ ID NO: 2)

| | |
|---|---|
| P-2: | 5′-CACCTCTCATTTCTCCCTGTTGACGCGCATCACTCG-3′ |

P-3 is assigned as the strand “b”, a common sequence in both adapter-A and adapter-B, and its sequence is:

| | | |
|---|---|---|
| P-3: | 5′-GATCCGAGTGATGCGCTAAG-3′ | (SEQ ID NO: 3) |

Ten pmol of P-1 and 10 pmol of P-3 are added into the digested cDNA solution from source A, and 10 pmol P-2 and 10 pmol P-3 are added into the digested cDNA solution from source B. Both solutions contain Tris-HCl (pH7.6), 6.5 mM MgCl₂, 0.5 mM ATP, 0.5 mM DDT and 2.5% polyethene glycol-800. The mixture is incubated at 70° C. for 10 min and slowly cooled down to 16° C. After T4 DNA ligase is added, ligation reaction is performed at 16° C. for 2 hours. These ligation products are used as the templates for the next PCR.

3. PCR Amplification of cDNA Fragments and the Preparation of Single-Stranded DNA.

The digested cDNA fragments from source-A and source-B are ligated with a corresponding adapter, respectively, and the two ligation products are equally mixed together as the template of PCR amplification. The sequence of 5′terminal region in each adapter is used as a primer of PCR. The first 21 bases from 5′-end in P-1 and P-2 are assigned as MP-1 and MP-2, respectively, and the mixture of MP-1 and MP-2 are used as PCR primers. The other primer for PCR is P53 gene-specific oligonucleotide, namely GSP, and labeled with biotin in the 5′-end. The mixture containing an equal amount of MP-1, MP-2 and GSP is employed as PCR primers. Ten μl of PCR solution contains 1 μl of templates, 1 pmol of each primer, 20 mM of Tris-HCl (pH 8.0), 50 mM of KCl, 0.2 mM of each of dNTPs and 1.25 U of DNA polymerase. PCR reaction is carried out at the thermal cycling conditions of 30 cycles at 94° C. for 30 s, 58° C. for 1 min and 72° C. for 30 s. The obtained products are biotinylated double-strand DNA, and are reacted with streptavidin-coated beads (Dynabeads M280) at room temperature for 30 min in the buffer of 5 mM of Tris-HCl (pH7.5), 0.5 mM of EDTA and 1.0 M of NaCl. After the reaction, the supernatant is discarded and 0.1 M of NaOH is added for the incubation at room temperature for 5 min. The beads are then washed and stored at the buffer of 5 mM of Tris-HCl (pH7.5), 0.5 mM of EDTA and 1.0 M of NaCl for future use. These beaded products are the mixture of single stranded DNAs from source-A and source-B.

4. Determination of Gene Expression Levels from Each Source by Bioluminometric Assay

Five pmol of MP-1 and 5 pmol of MP-2 are added into the single-stranded DNA sample (the products of beads in the step 3 above) containing 25 mM of Mg2⁺ and 5 mM of Tris (pH7.7). The mixture is incubated at 94° C. for 2 min and then placed at the environment of room temperature for cooling. 1˜5 μl of this template is added into 50˜100 μl of standard mixture for PPi assay. Sequencing reactions are carried out by dispensing dGTP and dCTP, respectively. In stead of dGTP and dCTP, ddGTP and ddCTP, or their analogues may also be used. The signal intensity obtained by adding dGTP represents the relative gene expression level in source A. The signal intensity obtained by adding dCTP represents the relative gene expression level in source B.

The standard mixture for PPi assay contains 0.1 M of Tris-acetate (pH7.7), 2 mM of EDTA, 10 mM of magnesium acetate, 0.1% bovine serum albumin (BSA), 1 mM of dithiothreitol (DTT), 3 μM of adenosine 5′-phosphosulfate (APS), 0.4 mg/ml of polyvinylpyrrolidone (PVP), 0.4 mM of D-luciferin, 200 mU/ml ATP sulfurylase, 2 U/ml of apyrase, 1 U of Klenow DNA polymerase without exonuclease activity, and a suitable amount of luciferase.

5. Instrument for the Detection

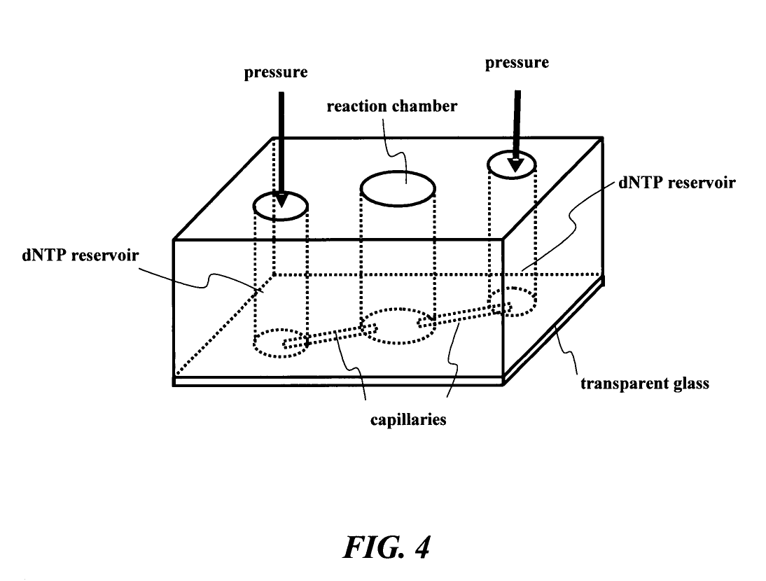

An instrument is designed for determining a single sample, and the key unit for the instrument is a reaction module as shown in FIG. 4. Capillaries are used for connecting the reaction chamber in the center with two dNTP reservoirs. The flow of dNTP or ddNTP from the reservoir into the reaction chamber starts by adding a pressure on the reservoir. The light released from the extension reaction goes through a transparent slide and is detected by a light sensor such as a photomultiplier tube (PMT) and a charge-coupled device (CCD) camera.

6. The Detection Results

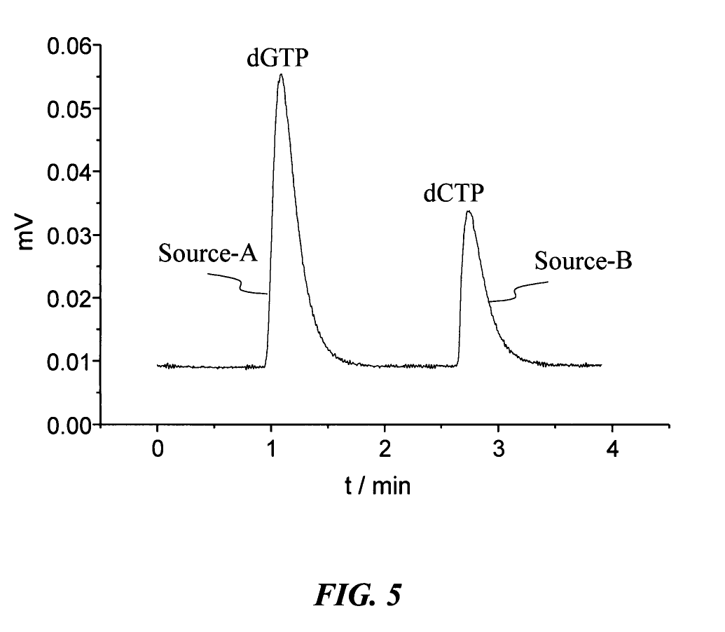

In the reaction module showed in FIG. 4, dGTP and dCTP are added into the two ddNTP reservoirs, respectively, and sample and PPi standard detection mixture are added into the reaction chamber in the center. A pressure is added on the top of dGTP reservoir and dCTP reservoir, respectively, by using a syringe. The sequencing signal from the reaction is illustrated in FIG. 5. The relative gene expression levels from two sources can be calculated from the signal intensities.

### EMBODIMENT 2

### Simultaneous Detection of Relative Expression Levels of 96 DIFFERENT Genes by Using a 96-Well Plate

In this embodiment, the expression levels of 96 genes in a disease group and a healthy group are determined simultaneously using a regular 96-well plate.

1. Preparation of Samples for Detection

In accordance with the method described in EMBODIMENT 1, after the extraction of mRNA from source-A and source-B, respectively, double-stranded cDNAs are prepared and digested by the restriction endonuclease Mbo I. The digested fragments are then ligated with DNA adapters corresponding to source-A and source-B, respectively, by ligase. 1˜5 μl of the ligated mixture is added into each well in 96-well plate to be a template of PCR amplification. PCR amplification is performed after MP-1, MP-2 and the gene-specific primer (GSP) are added into each well. The 5′-end of GSP primer is modified by biotin. The single-stranded DNA is prepared by the same procedure described in the EMBODIMENT 1. Finally, the mixture of MP-1 and MP-2 with the equal amount is added into every well in the plate as sequencing primers. The experimental procedure is the same as that in EMBODIMENT 1.

2. Instrument for the Detection

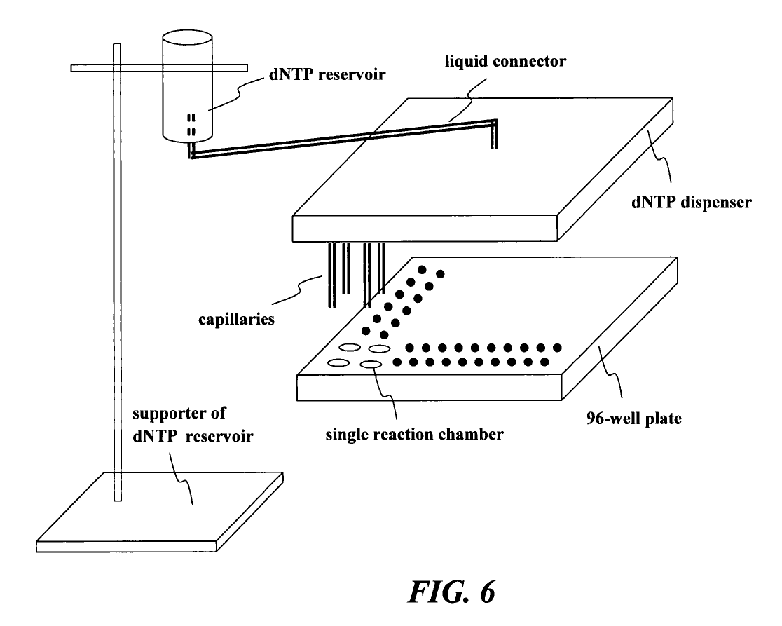

The key point of this EMBODIMENT 2 is to construct a device for detecting 96 samples in parallel. FIG. 6 depicts a schematic structure of the device using pressure difference for the injection. According to the dimension of a 96-well plate, capillaries are used to make two sets of liquid dispensers. Each of 96 capillaries in a dispenser is a dNTP addition header corresponding to a well in a 96-well plate.

One end of a capillary is connected with a reservoir of dNTP or ddNTP. The reservoir is above the 96-well plate. At the state of detection, headers of ddNTP addition in the dispenser are inserted into reaction mixtures by a lifter. When adding a pressure in dNTP reservoir, dNTP solution flows into reaction wells to trigger the incorporation reaction. PPi released during the reaction is quantitatively converted to ATP under the catalysis of ATP sulfurylase. The produced ATP drives the fluorescence production in the presence of luciferin and luciferase. A charge-coupled device (CCD) camera, PMT or photodiode array is used to detect the signals released from 96 wells. Of course, this device may also be used for detecting one gene in multiple samples simultaneously.

### EMBODIMENT 3

### Comparison of the Expression Level of One Given Gene Among Six Different Sources

Usually, a microarray chip is only used to determine gene expression levels from two different sources. If one source is added, an additional dye for labeling is needed. As a result, the detection cost increases. Conventionally, more than four kinds of dyes with different laser-excitation wavelengths are not used simultaneously. In the present invention, the pyrosequencing method is used to determine the gene expression levels from different sources, and a base sequence represents the source of a gene. Therefore, an increase of a source may not increase the detection cost.

As dATP is an analog of ATP, it produces a high background signal which severely interferes with the detection. Although an analog of dATP, dATPαS, can be used to replace dATP for the detection, the detection cost increases. In this invention, dATP is not employed. When comparing the gene expression levels from two different sources, a sequence of “cag” and “gac” are added into P-1 and P-2 for labeling the sources, respectively. Since at most three kinds of dNTPs, “g”, “c” and “t”, are possible for the sequencing, gene expression levels from at most three different sources can be determined by a single addition of a type of dNTP. As the PPi detection method in this invention has a very good quantitative capability, six kinds of different sequences located in the center of DNA adapters are designed to identify gene sources. The fourth base type in each sequence is designed as “T” in order to control further extension by dNTP. The sequences are as follows:
(1) cgat; (2) gcat; (3) agct; (4) gact; (5) cagt; and (6) acgt.

Besides dATP, three kinds of dNTP are added in the order of dTTP, dGTP, dCTP, dTTP and dGTP, and the total number of times added is seven. The relative signal intensities of six peaks observed in the sequencing spectrum are used to calculate the gene expression level from the source represented by each peak. Finally, the relative gene expression levels from each of sources are obtained readily by computer software based on the calculation method of simultaneous equations.

### EMBODIMENT 4

### Comparison of Average Gene-Expression Levels in Pooled Samples

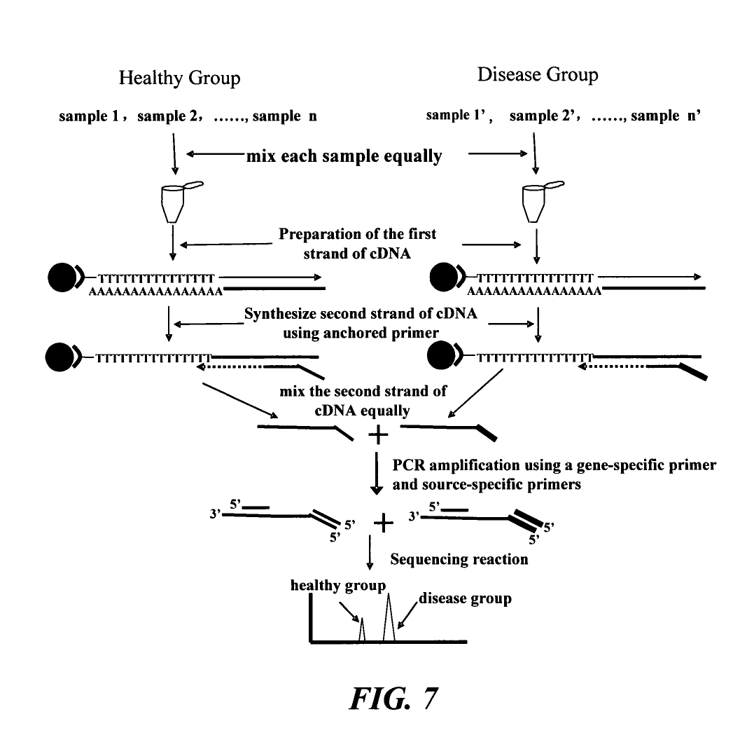

In this EMBODIMENT 4, the purpose is to determine the differences of average gene expression level between two groups, for example, a healthy group and a disease group. Provided that each of groups contains 100 cases, at least 100 detections are required by a conventional method such as a microarray assay, and then a result for each sample is obtained. Finally, the statistical results are obtained by analyzing the observed data through the computer software. In the present invention, 100 individual cases in a healthy group are pooled equally as a healthy group, and 100 individual cases in a disease group are pooled equally as a disease group. Then the relative gene-expression levels from these two pooled samples are detected just like the way for detecting two individual samples from two sources. The obtained results are the average gene-expression levels between two pooled samples. The disease-related genes are clarified by associating the gene expression levels with disease-production and disease-development. Compared with the gene chip technology, the efficiency for finding a disease-related gene is increased by 100 folds. With the gene chip method, 100 samples are needed to be determined. However, only a single detection is performed for 100 samples using the present method. Also the observed results are much more accurate. The procedure for the detection is described in FIG. 7.

The key points for determining the average gene-expression levels in pooled samples are as follows: (1) proportional PCR amplification on a small amount of cDNAs from two sources; and (2) an excellent quantification performance of signal detection. These requirements are satisfied by the present method. Thus, the average gene-expression levels from different pooled samples can be accurately compared. The detection speed and the reliability of results with this method are better than those with chip method.

Microsphere-based method is used for sample preparation. The first strand cDNA is synthesized after the hybridization of mRNA with polythymine primers fixed on microsphere's surface. The complementary strand of the first strand cDNA is synthesized by a gene-specific primer with an anchored sequence for identifying the source of each cDNA in the 5′-terminal region. In this EMBODIMENT 4, the anchored sequences are the first 23 bases from 5′ terminus in P-1 and P-2 of the EMBODIMENT 1, respectively. After the synthesis of cDNA is finished, DNA strands separated from beads are used as templates of PCR amplification. Finally, a biotinylated gene-specific primer and MP-1 and MP-2 of the EMBODIMENT 1 are used as primers for PCR amplification. The rest of the procedure is the same as that in the EMBODIMENT 1.

### EMBODIMENT 5

### Determination of Gene Expression Levels by Standard PPi Detection Mixture without the Addition of Apyrase

In pyrosequencing, a key is to use apyrase to degrade dNTP and ATP simultaneously for the successive sequencing reaction. In this invention, when determining gene expression levels from two or three sources, the dNTPs added for sequencing are of three different species. Thus, no interference occurs, and the added dNTP existing in the solution will not trigger the extension reaction when the next dNTP is added. On the other hand, it is not necessary to degrade ATP in the reaction mixture. As the linear range of ATP detection by luciferin-luciferase assay is very large, the signal intensities produced by ATP can be easily controlled in a linear range when adding another type of dNTP or two types of dNTPs.

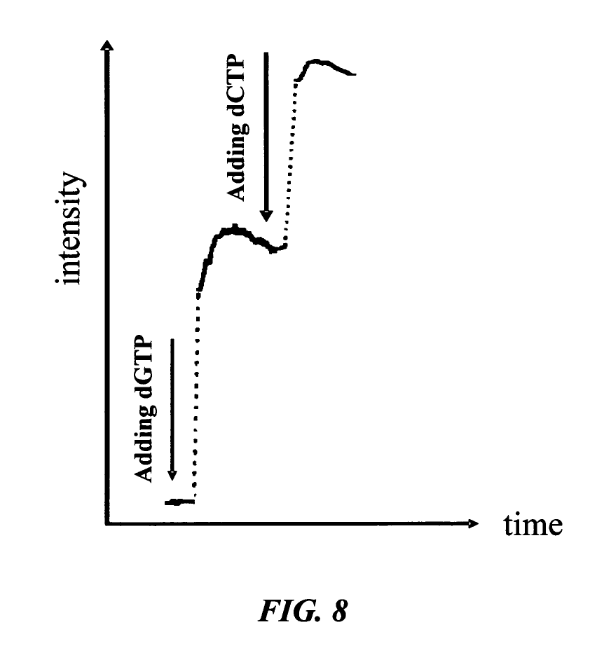

In this EMBODIMENT 5, the same sample of the EMBODIMENT 1 is determined by PPi detection mixture without the addition of apyrase. The results are showed in FIG. 8, which indicates that it is feasible to employ PPi detection mixture without the addition of apyrase.

What is claimed is:

1. A method for comparing gene expression level, characterized in that the method includes:
(a) labeling mRNA from different sources with a suitable method, and mixing the labeled mRNA fragments equally to obtain a template for polymerase chain reaction (PCR);
(b) performing a polymerase chain reaction using source-specific primers and a gene-specific primer; and
(c) detecting a sequence of amplified DNA fragments with bioluminescence analysis, a base type and a signal intensity in a sequencing profile representing a gene source and a relative expression level, respectively.

2. The method for comparing gene expression level according to claim 1, wherein the mRNA from different sources is an expressed mRNA of a given gene from different individuals of a species, or is an expressed mRNA of a given gene from different organs of an individual, or is an expressed mRNA of a given gene of a same species at different states of chemical stimulation or physical stimulation.

3. The method for comparing gene expression level according to claim 1, wherein the source-specific primers include an identical base species and base number but a different base order, each primer representing a gene source.

4. The method for comparing gene expression level according to claim 1, wherein the suitable method is a method to distinguish a gene source by a DNA fragment with a suitable length,
a first of the method including:
performing a reverse transcription-polymerase chain reaction (RT-PCR) to obtain complementary DNA (cDNA) fragments of a given gene in each source;
digesting cDNA into fragments with a suitable length using a restriction endonuclease; and
ligating each of the digested cDNA fragments with a selective adapter, a different adapter corresponding to mRNA from a different source;
a second of the method including:
synthesizing a first strand of complementary DNA (cDNA) fragments of mRNA samples from each source using polythymine primers fixed on microsphere's surface; and
synthesizing a complementary second strand of cDNA using anchored primers containing sequences corresponding to gene sources in a 5′-terminal region, a 5′-terminal region being used for identifying different sources of a given gene;
and a third of the method including:
preparing a first strand of the complementary DNA (cDNA) fragments of mRNA samples from each source by directly hybridizing anchored primers containing sequences corresponding to gene sources in a 5′-terminal region with mRNA; and
constructing of a 5′-terminal region of the anchored primers the same as that in the second of the method.

5. The method for comparing gene expression level according to claim 4, wherein the selective adapter is a cuneal dsDNA (double strand DNA) containing a part of sequences complementary to recognition sequences of the restriction endonuclease and can be fully ligated with restriction enzyme cutting ends in DNA fragment by a DNA ligase, a 5′ terminal region of one of the strands in the adapter containing a sequence specific to gene sources, a 3′ terminal region of the other strand in the adapter containing bases non-complementary to a opposite strand, or a 3′ end of the other strand in the adapter being modified to block ability of extension reaction by DNA polymerase, and the adapter having a structure of a “Y” shape consisting of two strands, one end of the adapter being divided into two branches due to no complementary bases, and the other end being formed of a shape of restriction enzyme cutting site.

6. The method for comparing gene expression level according to claim 4, wherein the part used for identifying gene sources in selective adapters and anchored primers includes identical base species and base number but different base order, each of the selective adapters having a same melting temperature, and each of the anchored primers including a same melting temperature.

7. The method for comparing gene expression level according to claim 1, wherein the bioluminometric assay is based on a quantitative determination of pyrophosphate released from an extension reaction.

8. The method for comparing gene expression level according to claim 7, wherein the extension reaction is polymerization of single-stranded PCR products annealed with a given primer or primer mixtures by DNA polymerase when a deoxynucleotide (dNTP) added in a given order, or a dideoxynecleoide (ddNTP) added in a given order, or an analog of dNTP or ddNTP added in a given order is complementary to the template.

9. The method for comparing gene expression level according to claim 8, wherein the single-stranded PCR products are obtained by treating the PCR products of claim 1 with a physical method or a chemical method, the physical method being to use a biotinylated primer for PCR amplification and then to prepare single-stranded DNAs by a solid phase method, and the chemical method being to use an enzyme for the digestion to prepare single-stranded DNAs.

10. The method for comparing gene expression level according to claim 7, wherein the extension reaction is polymerization of the PCR products of claim 1 treated by enzymes to degrade PPi produced during PCR reaction, excess dNTPs and excess primers, a single-strand binding protein (e.g. SSB) being added into the treated PCR products, the rest being performed in accordance with claim 8.


SEQUENCE LISTING (3 sequences)

SEQ ID NO: 1 - DNA, 36 bp, Artificial
  Chemically synthesized
ccccacttcttgttctctcatcaggcgcatcactcg

SEQ ID NO: 2 - DNA, 36 bp, Artificial
  Chemically synthesized
cacctctcatttctccctgttgacgcgcatcactcg

SEQ ID NO: 3 - DNA, 20 bp, Artificial
  Chemically synthesized
gatccgagtgatgcgctaag